(19) United States
(12) Patent Application Publication
(10) Pub. No.: US 2022/0348939 A1
(43) Pub. Date: Nov. 3, 2022

(54) CRISPR-Cas system for genome editing in Zymomonas mobilis, and applications thereof

(71) Applicant: HUBEI UNIVERSITY, Wuhan, Hubei (CN)

(72) Inventors: Shihui Yang, Wuhan, Hubei (CN); Wenfang Peng, Wuhan, Hubei (CN); Wei Shen, Wuhan, Hubei (CN); Yanli Zheng, Wuhan, Hubei (CN); Li Yi, Wuhan, Hubei (CN); Lixin Ma, Wuhan, Hubei (CN)

(21) Appl. No.: 17/295,045

(22) PCT Filed: Oct. 22, 2019
(86) PCT No.: PCT/CN2019/112556

§ 371 Date: May 19, 2021

(30) Foreign Application Priority Data
Jul. 30, 2019 (CN) 201910692112.0
Jul. 30, 2019 (CN) 201910692118.8
Jul. 30, 2019 (CN) 201910692120.5
Jul. 30, 2019 (CN) 201910692143.6

(51) Int. Cl. C12N 15/66; C12N 15/74; C12N 1/20

(52) U.S. Cl. CPC C12N 15/66; C12N 15/74; C12N 1/205; C12R 2001/01

(57) ABSTRACT

The invention belongs to the technical field of genetic engineering, and particularly to a type I-F CRISPR-Cas system based on Zymomonas mobilis (Z. mobilis) including: four CRISPR structural sequences and one cas gene cluster, wherein the cas gene cluster comprises casi gene, cas3 gene, csyl gene, csy2 gene, csy3 gene and csy4 gene, wherein the cast-3 gene is a fusion form fused by a cast gene and a cas3 gene. The purpose of the present invention is to use Z. mobilis as a model bacterium, using a CRISPR-Cas system encoded by the genome of the Z. mobilis and exogenous CRISPR-Cas12a system to build a genome editing platform so as to provide a set of powerful tools for carrying out basic and applied research in this bacterium and similar cells, and promoting the development of metabolic engineering, systems biology and synthetic biology.

### BACKGROUND OF THE PRESENT INVENTION

### Field of Invention

The present invention relates to the technical field of genetic engineering, and more particularly to an endogenous Type I-F CRISPR-Cas system based on Zymomonas mobilis (Z. mobilis), and a heterologous CRISPR-Cas12a system for genome editing and applications thereof.

### Description of Related Arts

In recent years, the use of microorganisms in metabolic engineering, systems biology and synthetic biology has made good progress, which provides an important theoretical basis for the rational design and construction of microbial cell factories, the use of living cells or enzymes for renewable biochemical production, such as the use of lignocellulose and other renewable substances to produce biofuels and to realize the industrialization of bio-smelting. Regeneration of biofuels is one of the effective means to solve the problems of resources, energy shortage and serious environmental pollution facing human beings.

Zymomonas mobilis (Z. mobilis) has the relevant characteristics of a satisfying microbial cell factory: (1) Z. mobilis can naturally produce alcohol and has a high tolerance to alcohol, which can use corn stover hydrolysate to produce 10.7% (v/v) of high-concentration bioethanol. (2) Z. mobilis is currently the only known microorganism that can metabolize glucose or fructose through the Entner-Doudoroff (ED) pathway to produce ethanol under anaerobic conditions. One molecule of ATP is produced with low biomass, so most of the carbon source (>95%) is converted into product ethanol, only about 2-2.6% of the carbon source is used for cell growth, and the target product yield is very high compared with the classical industrial ethanologen Saccharomyces cerevisiae. (3) The genome size is only about 2 Mbp, which has great advantages in carrying out genome reduction work and constructing the suitable synthetic cell factories. In addition, Z. mobilis can grow in a wide range of temperatures (24-45° C.) and pH range (3.5-7.5), and is recognized as a GRAS (Generally Recognized as Safe) strain.

Therefore, Z. mobilis can be used as a model production strain for renewable biofuels, which has attracted extensive attentions from researchers to analyze its physiological and genetic characteristics, to explore its metabolic network and regulatory mechanisms, and to rational design and construct recombinant strains. All these works need to make changes to genomic DNA sequences within cells, and efficient and accurate genome editing systems are urgently needed to fully investigate and develop Z. mobilis as suitable industrial microbial cell factories for economic biochemical production.

Conventional genetic methods generally use the DNA recombination system in the host to make changes, but usually can only be done individually, which takes a long period of time and the efficiency is low. Obviously, it cannot meet the requirements of research such as the construction and dynamic control of complex metabolic pathways. Moreover, for each mutation of the target gene, specific selection markers need to be introduced, and there will be some problems such as the limitation of available selection markers, and the potential bio-security risks of the introduction of antibiotic markers. In addition, in order to improve the efficiency of DNA recombination in cells, sequence-specific nucleases can be used to site-specific cut the target gene, promote the recombination of genome and donor DNA, and introduce mutations at site to achieve precise editing of the genome. For example, Zinc finger nucleases (ZFNs) and transcription activator-like effector nucleases (TALENs) have been successfully used for site-specific shearing of the genome. However, the shearing of each target site requires the modification of the above proteins, and the experimental procedure is relatively cumbersome, time-consuming, and expensive.

Since 2013, genome editing technology based on CRISPR-Cas9 and CRISPR-Cas12a (aka CRISPR-Cpf1) system has been widely used in various organisms or cells including human cells. Compared with ZFN and TALEN technology, CRISPR-Cas technology has an apparent advantage as follows. Targeting of different target DNAs does not require protein modification, but only needs to simply change the single mediating guide RNA (gRNA) sequence, which is easy to operate. However, as of now, no related applications have been successfully implemented in Z. mobilis, the main reasons may be as follows. 1) Both Cas9 and Cas12a are large nuclease proteins (more than 1,000 amino acids) with multiple domains, which are found in prokaryotic cells and there is a limitation for effective transfer in. 2) Exogenously expressed Cas9 or Cas12a and other nucleases may be inactive, or active but produce cytotoxicity to the host especially the Cas9 nuclease.

### SUMMARY OF THE PRESENT INVENTION

In view of the problems in the conventional art, the present invention provides a genome editing system for Z. mobilis, and applications thereof, which includes an endogenous Type I-FCRISPR-Cas system based on Z. mobilis and a heterologous CRISPR-Cas12a system. The purpose of the present invention is to use Z. mobilis subsp. mobilis ZM4(ZM4) as a model strain to build a genome editing platform using the type I-F CRISPR-Cas system encoded by its own genome and a heterologous CRISPR-Cas system, so as to provide a set of powerful tools for carrying out basic and applied researches of metabolic engineering, systems biology and synthetic biology in this strain and similar cells.

The present invention is achieved by the technical solutions as follows. An endogenous Type I-F CRISPR-Cas system based on Zymomonas mobilis (Z. mobilis), comprises: four CRISPR structural sequences and one cas gene cluster, wherein the cas gene cluster comprises a cas1 gene, a cast-3 gene, a csy1 gene, a csy2 gene, a csy3 gene and a csy4 gene, wherein the cast-3 gene is a fusion form fused by a cast gene and a cas3 gene.

The present invention is realized in this way, based on Z. mobilis endogenous Type I-F CRISPR-Cas system, which comprises the cas gene cluster of the cast, the cas3, the csy1, the csy2, the Csy3 and the csy4 genes, of which cas3 gene is a fusion of the cas2 and the cas3 genes.

Genome editing system comprises: an editing system based on exogenous CRISPR-Cas12a system, including the editing plasmid carrying a targeting sequence of guide RNA (gRNA) primer sequence, an artificial CRISPR expression unit and plasmids edited by a donor DNA sequence, and a recombinant Z. mobilis strain containing an inducible gene encoding nuclease Cas12a.

Furthermore, the recombinant Z. mobilis containing inducible nuclease Cas12a is obtained by integrating exogenous nuclease Cas12a into Z. mobilis containing the CRISPR-Cas system as recited in claim 1, and the expression of Cas12a nuclease is controlled through a tetracycline-inducible promoter Ptet.

Furthermore, the artificial CRISPR expression unit comprises a constitutive promoter PJ23119, a repeat sequence, and two restriction sites.

Furthermore, the PAM sequence of the editing system is TTTN.

A genome editing system for endogenous Type I-F CRISPR-Cas system based on the Z. mobilis, comprising: an editing plasmid carrying a targeting sequence of gRNA primer sequence, an artificial CRISPR expression unit, and plasmids edited by a donor DNA sequence.

Furthermore, the artificial CRISPR expression unit comprises a leader sequence, a CRISPR cluster and a terminator, the CRISPR cluster comprises two enzyme cleavage sites inserted between two repeat sequences.

Furthermore, the leader sequence, CRISPR cluster and terminator sequence are shown in SEQ ID NO: 34-SEQ ID NO: 36 respectively

Furthermore, the PAM Sequence of the Editing System is NCC

Application of the endogenous Type I-F CRISPR-Cas system based on Z. mobilis, and the genome editing system for exogenous CRISPR-Cas12a system in Z. mobilis for gene knockout, insertion, site-directed DNA mutation, simultaneous editing of multiple gene sites, deletion of large genome fragments, elimination of endogenous plasmids, or detection of protein active sites.

In summary, the advantages and positive effects of the present invention are:

The present invention uses the Z. mobilis model strain ZM4 as a material, develops its endogenous CRISPR-Cas system as a gene editing tool, and performs a series of efficient genetic operations. This technology has the following beneficial effects: (1) It can effectively avoid the cytotoxicity of foreign Cas nucleases to the host. (2) The host's own CRISPR-Cas system has complete functions, and can process mature crRNA to mediate the effect of Cas complex to cut the target DNA sequence. (3) There is no problem of transforming large proteins into the host. (4) The editing purpose of deleting large fragments of the genome of the strain can be achieved. Therefore, cloning different mediating sequences into the same CRISPR cluster enables simultaneous editing of multiple target sites. Compared with the traditional genetic operation method, using the CRISPR-Cas system, (1) can continuously cut the pre-edited target sequence, which has a strong positive selection pressure, and does not require additional selection markers avoiding what the traditional genetics operation often encountered in the method: limited selection markers available, and the potential safety hazards posed by the introduction of antibiotic markers; (2) The cycle of gene editing is greatly decreased. Taking single-site editing as an example, the conventional genetic manipulation method requires at least 15 days to obtain the purified target strain, while only 3 days are required using the CRISPR-Cas system; for multi-site editing, the difference is greater, the conventional genetic manipulation method can only be accumulated based on the editing of a single site, that is, at least 30 days for 2 sites, at least 45 days for 3 sites, and so on, using the CRISPR-Cas system still only needs 3 days (one editing cycle). The invention has realized the simultaneous editing of at least 3 points.

The invention breaks the limitation of low efficiency of exogenous CRISPR-Cas9 genome editing in such strains due to the cytotoxicity of Cas9 nuclease, achieves rapid and efficient knockout of multiple genes in this strain, and promotes the development of metabolic engineering, systems biology and synthetic biology.

In addition, the present invention has also developed a genome editing system in Z. mobilis based on the exogenous CRISPR-Cas12a system, and explored its applications in endogenous plasmid elimination, gene knockout, gene mutation and gene insertion. The system has the following technical advantages. (1) The operation is simple. Because CRISPR-Cas12a can process itself to form crRNA, it only needs to assemble the target site sequence and provide an appropriate repair template for gene editing. (2) High positive rate and seamless editing. CRISPR-Cas12a system can continuously cut the target sequence, with positive selection pressure, no additional resistance selection markers for the capability of continuous genome editing, and avoid the safety risks caused by the introduction of resistance genes. (3) Wide range of applications. The CRISPR-Cas12a system can be used for a variety of gene editing methods such as: gene knockout, gene knock-in, and site-directed mutation. (4) The process is simple, and the time period is short, which greatly reduces the workload of prokaryotic genome editing.

### BRIEF DESCRIPTION OF THE DRAWINGS

FIG. 1 is a CRISPR-Cas system encoded by Z. mobilis;

FIG. 2 is the C2S7 and C3S4 sequence;

FIG. 3 is the result of in vivo shear activity detection of CRISPR-Cas system;

FIG. 4 is an artificial CRISPR expression unit;

FIG. 5 is a schematic diagram of the principle of guide RNA construction into a vector during gene knockout;

FIG. 6 is the result of PCR cloning in gene knockout;

FIG. 7 is the result of PCR product sequencing in gene knockout;

FIG. 8 is a schematic diagram of the principle of point mutation of DNA sequences;

FIG. 9 is the result of PCR cloning of the DNA sequence with point mutation;

FIG. 10 is the result of sequencing the PCR products of the point mutation DNA sequence;

FIG. 11 is a schematic diagram of the principle of site-directed mutation DNA sequence;

FIG. 12 is the result of PCR cloning of site-directed mutation DNA sequence;

FIG. 13 is the result of PCR product sequencing of site-directed mutation DNA sequence;

FIG. 14 is a multi-point simultaneous editing process mediated by endogenous CRISPR-Cas;

FIG. 15 is a schematic diagram of the process of cloning an artificial CRISPR cluster into an editing plasmid;

FIG. 16 is the results of electrophoresis of PCR products of transformant colonies;

FIG. 17 is a schematic diagram of statistical analysis of the transformant editing results;

FIG. 18 is the sequencing results of transformants;

FIG. 19 is a schematic diagram of the principle of deleting a large-fragment sequence;

FIG. 20 is the colony PCR positive clone results;

FIG. 21 is the result of transformant sequencing;

FIG. 22 is a product electrophoresis diagram of the endogenous plasmid elimination experiment;

FIG. 23 is a product electrophoresis diagram of a point mutation experiment;

FIG. 24 is the product sequencing results of the point mutation experiment;

FIG. 25 is a schematic diagram of the principle of gene knockout editing;

FIG. 26 is a product electrophoresis diagram of gene knockout experiments;

FIG. 27 is the product sequencing results of gene knockout experiments;

FIG. 28 is a schematic diagram of the principle of gene insertion editing;

FIG. 29 is a product electrophoresis diagram of gene insertion experiments;

FIG. 30 is the product sequencing results of gene insertion experiments;

FIG. 31 shows the results of flow cytometry in gene insertion experiments.

### DETAILED DESCRIPTION OF THE PREFERRED EMBODIMENT

The present invention will be further described in detail below in conjunction with examples. The equipment and reagents used in the examples and test examples can be obtained from commercial sources unless otherwise specified.

### Example 1 Construction of Endogenous CRISPR-Cas Genome Editing System of Zymomonas mobilis

(1) Systematic Analysis of CRISPR-Cas System in Z. mobilis Genome

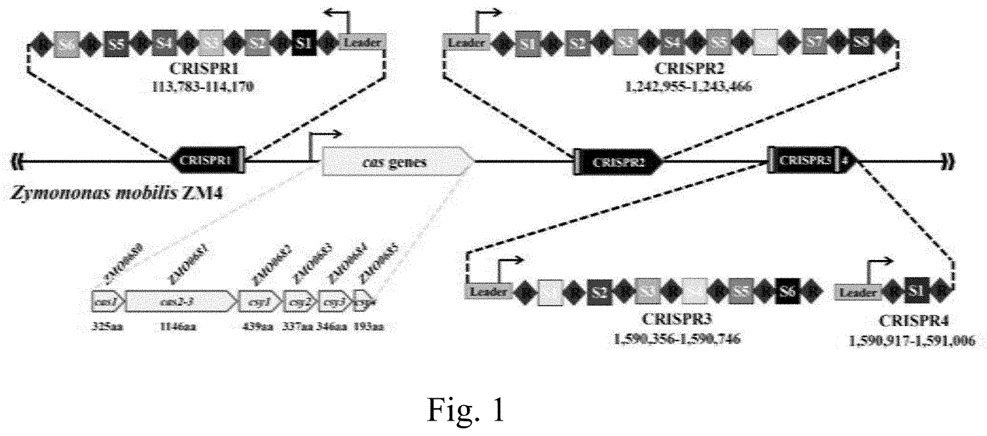

Taking Z. mobilis ZM4 as a model strain, genome sequence data of Z. mobilis ZM4 was analyzed. The results showed that the genome of the strain encodes 4 CRISPR structural sequences, which are named CRISPR1-CRISPR4 sequentially according to their arrangement order in the genome, see FIG. 1. CRISPR1 occupies the 113,783-114,170 region of the genome and contains 7 repeat sequences; CRISPR2 occupies the 1,244,355-1,245,866 region and contains 9 repeat sequences; CRISPR3 occupies the 1,598,754-1,599,144 region and contains 7 repeat sequences. CRISPR4 is composed of 2 repeats and 1 spacer, occupying 1,595,315-1,599,403 regions. CRISPR2-4 is on the same strand, while CRISPR1 is on the complementary strand. The repeats in these CRISPR structures are all conserved 28 bp sequences, and the spacer length is 32 or 33 bp, of which 32 bp account for 70%. The genome also encodes a cas gene cluster, comprising cas1, cas3, csy1, csy2, csy3 and csy4 genes, of which cast and cas3 form an operon, and all csy genes are arranged in the form of an operon. In the above results, the cas3 gene is a fusion of cast and cas3 genes, and is a hallmark feature of the I-F CRISPR-Cas system.

(2) Detection of the In Vivo Shearing Activity of the Endogenous I-F CRISPR-Cas System of Z. mobilis

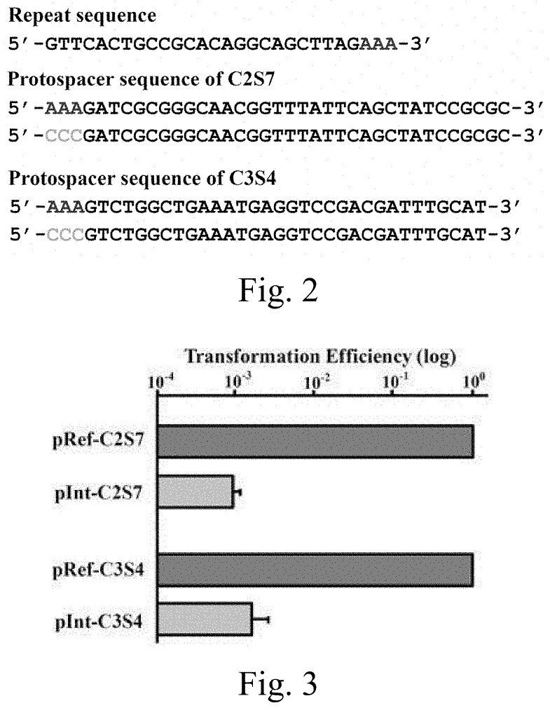

In order to test whether the type I-F CRISPR-Cas system in Z. mobilis ZM4 strain can cleave DNA in vivo under the guidance of crRNA, according to the results of transcriptomic analysis, the spacer? in CRISPR2 (C2S7) and the spacer4 in CRISPR3 (C3S4) were selected with 5′-CCC-3′ PAM added to the 5′end of the sequence, see FIG. 2. The specific operation is as follows: the Z. mobilis-E. coli shuttle vector pEZ15Asp is double digested with Xba I and EcoR I, the digestion system is the system 1: pEZ15Asp, 2-3 μg; Xba I, and EcoR I each 1 μL; Buffer, 2 μL; supplement H₂O to 20 μL. Carrier double digestion conditions: 37° C. for 3-4 h. After annealing with the primers of C2S7 and C3S4, they were ligated with T4 DNA ligase. The annealing system is System 2: Buffer, 1 μL; forward primer and reverse primer 1 μL each; supplement H₂O to 10 μL. Annealing procedure: hold at 95° C. for 5 minutes, and then anneal at room temperature. T4 DNA ligase system is system 3: linearized vector 100-200 ng; primer 1 μL after annealing; T4 DNA ligase 1 μL; buffer 2 μL; supplement H₂O to 20 μL. Ligation reaction procedure: keep at 22° C. for 2 h. It was transformed into E. coli DH5a by 42° C. standard heat shock transformation method for plasmid amplification, and then the colonies were verified by PCR. The PCR system was system 4: PCR mix, 5 μL; forward primer and reverse primer were 0.5 μL each; Template 1 μL; supplement H₂O to 10 μL. The PCR program is: Step 1, 98° C., 3 min; Step 2, 98° C., 10 s; Step 3, 55° C., 15 s; Step 4, 72° C., 30 s; Step 2-Step 4 cycle 25 times; Step 5, 72° C., 2 min. The constructed plasmids are all verified by Sanger sequencing. At the same time, the 5′plus 5′-AAA-3′ sequence spacer was inserted into the vector to obtain the corresponding reference plasmid. The shuttle vector pEZ15Asp contains the gene encoding spectinomycin resistance.

The sequences of C2S7 and C3S4 primers are shown in SEQ ID NO:1-SEQ ID NO:8.

The extracted plasmid was used to electroporate ZM4. The electroporation method is a general standard method in the art, and will not be repeated here.

It is predicted that after transforming the plasmids to the host separately, if the type I-F CRISPR-Cas system is active, the inserted proto spacer will be cut and screened on the RM medium plate containing 100 μg/mL spectinomycin, which will cause a few colonies formed on the surface of the plate; a large number of colonies were formed on the plate transformed with the reference plasmid.

The experimental results are shown in FIG. 3. The efficiency of transforming the interference plasmid is 103 times lower than that of the reference plasmid, which shows that the crRNA expressed in CRISPR on the genome mediates the DNA nuclease activity of the Type I-F system to cut the protospacer in the interference plasmid. To determine that the system can be used for targeted targeting and cleavage of DNA sequences.

(3) Composition of Genome Editing Plasmid

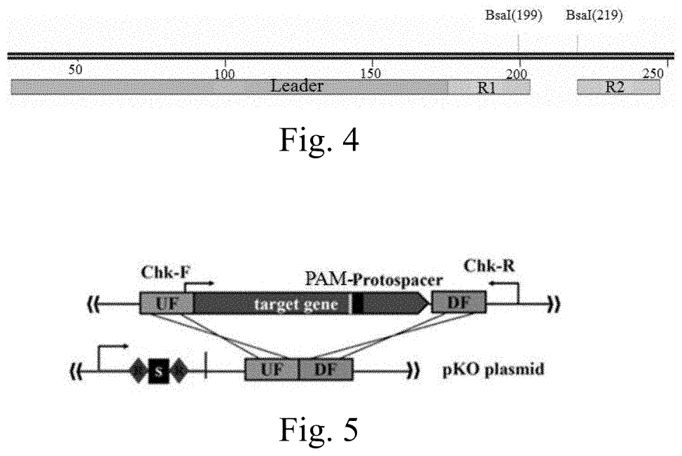

An artificial CRISPR expression unit was constructed on the plasmid pEZ15Asp, as shown in FIG. 4, consisting of the promoter leader sequence, CRISPR cluster and terminator. The artificial CRISPR expression unit was artificially synthesized by Genscript Inc. The artificial CRISPR cluster includes two Bsa I cleavage sites inserted between two repeats. The second is the supply of donor DNA. The design of the donor DNA is different according to the different genome editing forms, but the donor DNA is amplified and connected by fusion PCR technology. In this embodiment, the leader sequence, the gRNA module sequence, and the T7 terminator sequence are shown in SEQ ID NO: 34-SEQ ID NO: 36, respectively.

In the application of gene knockout, the sequences of about 300 bp upstream and downstream of the target gene are amplified and connected. In the application of site-directed gene mutation, the mutation site is introduced into the primer and the mutation is introduced into the donor DNA. The purified donor DNA is cloned into the corresponding editing plasmid for transformation.

### Example 2 Construction of Z. mobilis CRISPR-Cas12a Genome Editing System

In this example, gene encoding the nuclease Cas12a derived from Francisellanovicida was integrated into the ZMO0038 site in the Z. mobilis ZM4 genome by homologous recombination, and an inducible promoter Ptet was used to control nuclease expression level, so as to construct the recombinant strain ZM-Cas12a.

The specific construction process is as follows:

(1) Construction of Recombinant Plasmid

PCR was used to amplify the Cas12a gene sequence, resistance selection marker (spectinomycin), inducible promoter gene sequence (tetracycline-induced promoter), gene sequence upstream and downstream of the insertion site, and reverse amplification of pUC57 for integration vector sequence. The PCR amplification program was set as follows: performing pre-denaturation at 98° C. for 2 min; performing denaturation at 98° C. for 10 s, annealing at 55° C. for 10 s, performing extension at 72° C. according to the length of the fragment set at 10 s per kb, performing a total of 30 cycles; keeping at 72° C. for 5 minutes after the end of the cycle reaction; purifying the product and storing at −20° C. The PCR amplification condition system is system 5: 0.5 μL each of 10 μM forward and reverse primers; 10 μL of Primer STAR DNA Polymerase (Takara); X μL of Template (5-10 ng); supplemented with H₂O to 20 μL.

Among them, the templates for amplifying Cas12a and the inducible promoter fragments are both synthetic sequences, the Cas12a gene sequence is shown in SEQ ID NO: 69, and the promoter Ptet sequence is shown in SEQ ID NO: 70. The amplification of the spectinomycin resistance gene comes from vector pEZ15A. The template for the amplification of the upstream and downstream gene sequences comes from the genomic DNA (gDNA) of Z. mobilis ZM4. The template for reverse amplification of pUC57 is the pUC57 vector. See SEQ ID NO: 71-SEQ ID NO: 82 for primer sequences.

Mix the obtained fragments and vector in a 3:1 molar ratio, according to System 6 (DNA fragment, 0.12 pM; Vector, 0.04 pM; 10×Buffer 4 (Thermo), 0.5 μL; T5 Exonuclease, 0.5 U; supplement H₂O to 5 μL). After preparation, let stand on ice for 5 minutes, and then add chemical-competent cells to carry out chemical transformation. Screening with spectinomycin-resistant plates, single colonies were picked and verified by colony PCR with universal M13 primers (PCR amplification program setting: 98° C. pre-denaturation for 3 min; 98° C. denaturation for 10 s, 55° C. annealing for 10 s, Extended 80 s at 72° C. for a total of 30 cycles), and the band size was consistent with the expected verification by sequencing.

Afterwards, it is transformed and cultivated according to the general electroporation method. After the colony grows, the recombinant strain is tested by colony PCR. The PCR amplification system is system 7: 10 μM forward and reverse primers 0.3 μL each; 2×T5 Super PCR Mix (Tsingke), 5 μL; Template, X μL; supplement H₂O to 10 μL. The PCR amplification procedure is the same as the above procedure when constructing the recombinant plasmid. Strains with the same band size as expected were verified by sequencing, and the correct strains were kept for later use.

(2) Construction of Editing Plasmid

The editing plasmid uses pEZ15a as the vector backbone to construct an artificial expression unit of crRNA, which is expressed by a 19-nt repeat sequence and a 23-nt leader sequence under the control of a constitutive promoter PJ23119, in which two are inserted after the repeat sequence A Bsa I cleavage site facilitates insertion of the guide sequence. The specific construction process is as follows: PJ23119 (TTGACAGCTAGCTCAGTCCTAGGTATAATGCTAGC), 19-nt repeat sequence (AATTTCTACTCTTGTAGAT) and two Bsa I cleavage sites (GGAGACCGAGGTCTCA) are connected in series and assembled on pEZ15a vector. This process is completed by a tripartite company.

(3) After obtaining the plasmid containing the artificial CRISPR expression unit, design the donor DNA, construct the targeting plasmid according to the specific editing needs, and integrate the donor DNA into the targeting plasmid, and then transfer the targeting plasmid into competent cells for editing. Finally, the effect of editing is verified by colony PCR and sequencing.

### Example 3 Application of Z. mobilis CRISPR Genome Editing System Forgene Knockout

1. The genome editing system is selected according to the target gene and its editing type. In this example, ZMO0028 in the Z. mobilis ZM4 genome is used as the target site to knock it out, and the CRISPR-Cas12a system described in Example 2 is selected. The principle is shown in FIG. 25. The PAM site is selected from the target gene. The sequence of 23 bp downstream of the TTTN is used as the target guide sequence for constructing the gRNA in the target plasmid to guide the cleavage of the target site by the nuclease. The forward primer is 5′-AGAT+(target sequence)-3′, and the reverse primer is 5′-TGAC+(target sequence complementary sequence)-3′. See SEQ ID NO: 102 and SEQ ID NO: 103 for primer sequences.

2. Construction of Target Plasmid

The gRNA primer sequence is ligated to the editing plasmid containing the CRISPR expression unit prepared in Example 2: First, the vector is linearized with the restriction enzyme Bsa I (plasmid 2 μs; 10×buffer, 5 μL; enzyme 1 μL; supplement H₂O to 50 μL. Incubate at 37° C. for 3-4 h, and then anneal the gRNA primer pair (1 μL of each 10 μM primer is added to make up to 10 μL with water and denatured at 95° C. for 5 min, then cooled to room temperature for later use). The annealed product and the linearized vector are ligated using T4 DNA ligase (Annealed oligonucleotide, 0.5 μL; vector, 10 ng; 10×T4 DNA Polymerase Buffer, 1 μL; T4 DNA Polymerase, 0.2 μL; supplemented with H₂O to 10 μL). Then, it is transferred into the E. coli DH5a by a general chemical transformation method in the art for plasmid construction, and the recombinants are screened by colony PCR and finally verified by Sanger sequencing.

3. Construction of Editing Plasmid

The sequences of about 700 bp each on the upstream and downstream of the target gene were selected, and their DNA fragments were amplified by PCR, respectively. The PCR system and procedure are the same as in Part (1) of Example 2. The target vector constructed in the previous step is amplified by reverse PCR using primers. The PCR amplification system and procedure are the same as those in part (1) of Example 2 and the primers are described below. The fragments and vectors were then ligated by standard Gibson assembly methods, and then transferred into E. coli DH5a for plasmid construction, and recombinants are screened by colony PCR and finally verified by sequencing. The reverse amplified sequence is shown in SEQ ID NO: 104-SEQ ID NO: 105. See SEQ ID NO: 106-SEQ ID NO: 109 for the amplification of the upstream and downstream primer sequences.

4. Transformation of Editing Plasmid

Take about 500 ng of the editing plasmid constructed in step 3, and transform into competent cells of the recombinant bacteria containing Cas12a prepared in Example 2 by a general electroporation method. After the electroporation was completed, 1-mLRM medium was supplemented and then cultured at 30° C. for 4-6 h, about 200-50 ii g/mL chloramphenicol-resistant plate and culture at 30° C. 2-3 days.

5. Screening of Recombinant Bacteria

After the colonies grow out, the recombinant strains are subjected to colony PCR detection using the corresponding specific primers, respectively. The system and conditions are the same as those for recombinant bacteria detection in Example 2. The detection primer sequences are shown in SEQ ID NO: 110-SEQ ID NO: 111.

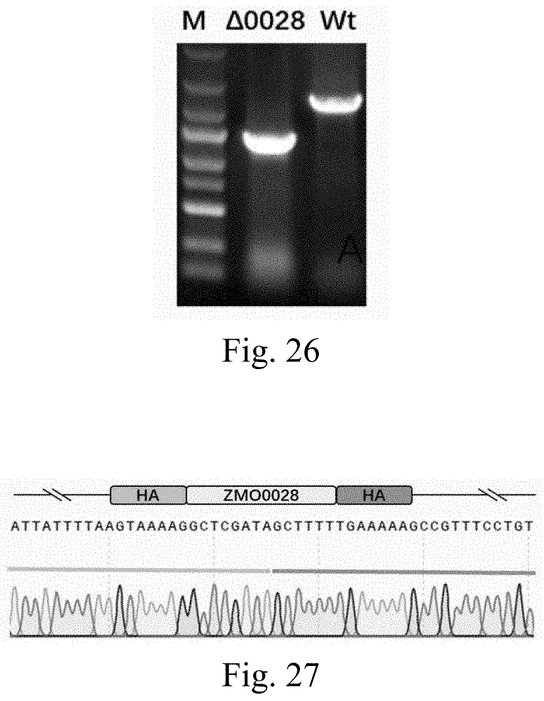

The results of PCR electrophoresis and sequencing results are shown in FIG. 26 and FIG. 27, indicating that the CRISPR-Cas12a system can be targeted to knock out genes, and the sequencing results show that the knocked-out genes are consistent with the design, indicating that this method is an accurate gene knockout method.

Similarly, for other types of target gene knockout, the CRISPR-Cas system in Example 1 can also be selected. The ZMO0038 gene encoded by Z. mobilis genome is used as the target gene for the description below.

From the target gene sequence ZMO0038, intercept any 32 bp sequence immediately downstream of 5′-CCC-3′ as gRNA, which can be located on any strand of the genome, and design primer sequences see SEQ ID NO: 9-SEQ ID NO: 10.

The gRNA primer sequence was constructed on the vector, see FIG. 5: The above plasmid containing the artificial CRISPR expression unit is linearized with Bsa I, annealed to the gRNA primer and ligated with T4 DNA ligase, and then transformed into E. coli DH5a for plasmid amplification Then, colony PCR is performed on the transformants, and the constructed plasmids are verified by sequencing. The experimental conditions are the same as above. For colony PCR verification primer sequence, see SEQ ID NO: 11 and SEQ ID NO: 10.

The donor DNA sequence selects the sequence of about 300 bp on the upstream and downstream of the target gene, and uses fusion PCR technology (PCRmix, 10 μL; forward and reverse primers 1 μL; template 1 and 2 each 1 μL; supplement H₂O to 20 μL. The PCR program is: 98° C., 3 min; 98° C., 10 s; 55° C., 15 s; 72° C., 30 s; 25 cycles; 72° C., 2 min, amplify and connect. Double digest the vector constructed in the previous step with Xba I and EcoRI (same as Example 1), and then transform E. coli DH5a with the donor DNA sequence assembled by Gibson assembly method, and then perform colony PCR verification on the transformants, wherein the method is the same as example 1. The constructed editing plasmids are all verified by sequencing. The donor DNA primer sequences are shown in SEQ ID NO: 12 and SEQ ID NO: 15.

The editing plasmid constructed and confirmed is transformed into ZM4 competent cells by universal electroporation method, and primer 0038-check-F (SEQ ID NO: 16) and 0038-check-R (SEQ ID NO: 17) are used to perform colony PCR screening, and positive clones wherein the conditions are the same as above.

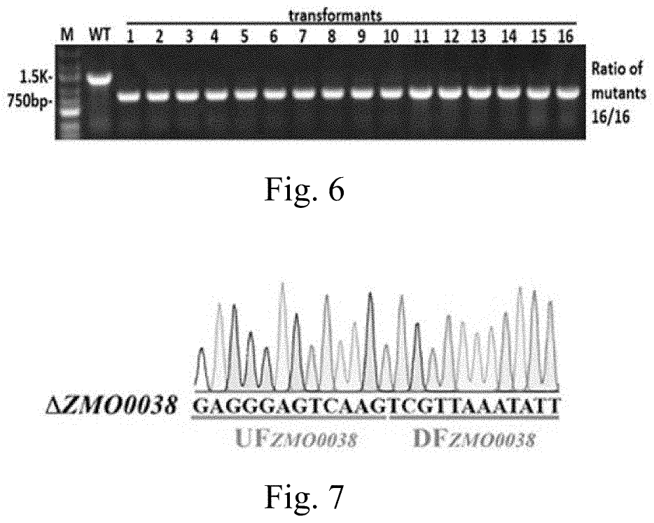

The result of positive cloning is shown in FIG. 6. The efficiency of ZMO0038 gene knockout reached 100%, which fully shows that the invention is an efficient gene editing method. Using the above PCR products for Sanger sequencing analysis, the results are shown in FIG. 7. Comparing the sequencing results with the wild-type reference genomic sequence, it was found that the gene editing method was completely in accordance with the experimentally designed scheme, further illustrating that the invention is an accurate gene editing method.

### Example 4 Application of Z. mobilis CRISPR Genome Editing System for Gene Insertion

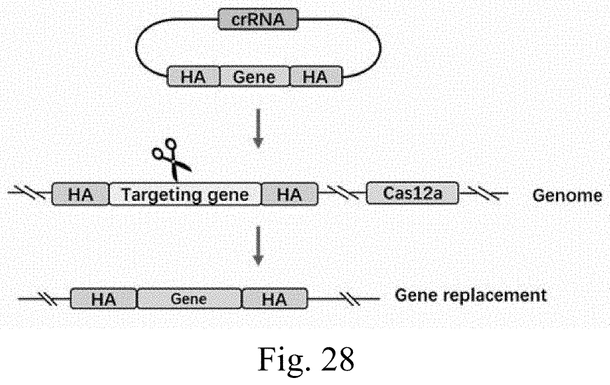

1. The genome editing system is selected according to the target gene and its editing type. In this example, ZMO0028 is used as the target site, the reporter gene mCherry is inserted into the genome, ZMO0028 is replaced, and the CRISPR-Cas12a system described in Example 2 is selected. The principle is shown in FIG. 28. The sequence of 23 bp downstream of the TTTN site of the PAM site is selected from the target gene as the target guide sequence of the gRNA in the construction of the target plasmid to guide the cleavage of the target site by the nuclease. The forward primer is 5′-AGAT+(target sequence)-3′, and the reverse primer is 5′-TGAC+(target sequence complementary sequence)-3′. The sequence of the gRNA primer is shown in SEQ ID NO: 112-SEQ ID NO: 113.

2. Construction of Target Plasmid

The gRNA primer sequence is ligated to the plasmid containing the CRISPR expression unit prepared in Example 2: First, the vector is linearized with restriction enzyme Bsa I, and then the gRNA primer pair was annealed. The annealed product is linear. The recombinant vector is ligated using T4 DNA ligase, and then transferred into E. coli strain DH5a for plasmid construction. The recombinants are screened by colony PCR and finally verified by sequencing. Specific experimental operation is the same as in Example 3.

3. Construction of Editing Plasmid

The donor DNA sequence is constructed on the target vector: the sequence of about 700 bp each upstream and downstream of the target gene and the mCherry expression element are amplified by PCR, and the PCR system and procedure are the same as in Example 2. The target vector constructed in the previous step is amplified by reverse PCR using primers, and then the fragments and the vector are connected by the method of Gibson assembly, in which the mCherry expression element was sandwiched by the upstream and downstream homology arm fragments, and then transferred to the E. coli strain DH5α, the recombinants containing the constructed plasmid are screened by colony PCR and finally verified by sequencing.

The primer sequences for reverse amplification are shown in SEQ ID NO: 114-SEQ ID NO: 115.

See the sequence of SEQ ID NO: 116-SEQ ID NO: 119 for the amplification of the upstream and downstream primer sequences.

The primer sequence of the amplified reporter gene expression element is shown in SEQ ID NO: 120-SEQ ID NO: 121.

4. The editing plasmid was electroporated and cultured, and the experimental method is the same as in Example 3.

5. The experimental method of recombinant strain screening is the same as in Example 3, and the primer sequences are shown in SEQ ID NO: 122-SEQ ID NO: 123.

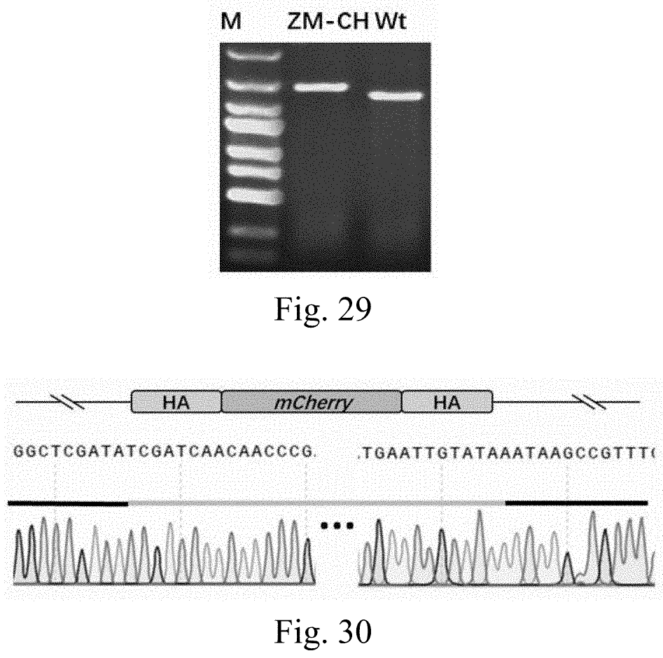

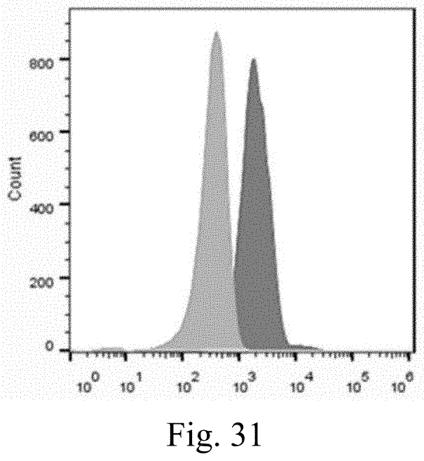

The results are shown in FIG. 29 and FIG. 30. The results show that the CRISPR-Cas12a system can be directed to insert genes, and sequencing results show that the gene was knocked-out as expected. In addition, after the insertion of the reporter gene, the flow cytometer was used for detection. The results are shown in FIG. 31. The results indicate that the reporter gene can be normally expressed at the insertion site. It shows that this method is an accurate gene insertion method.

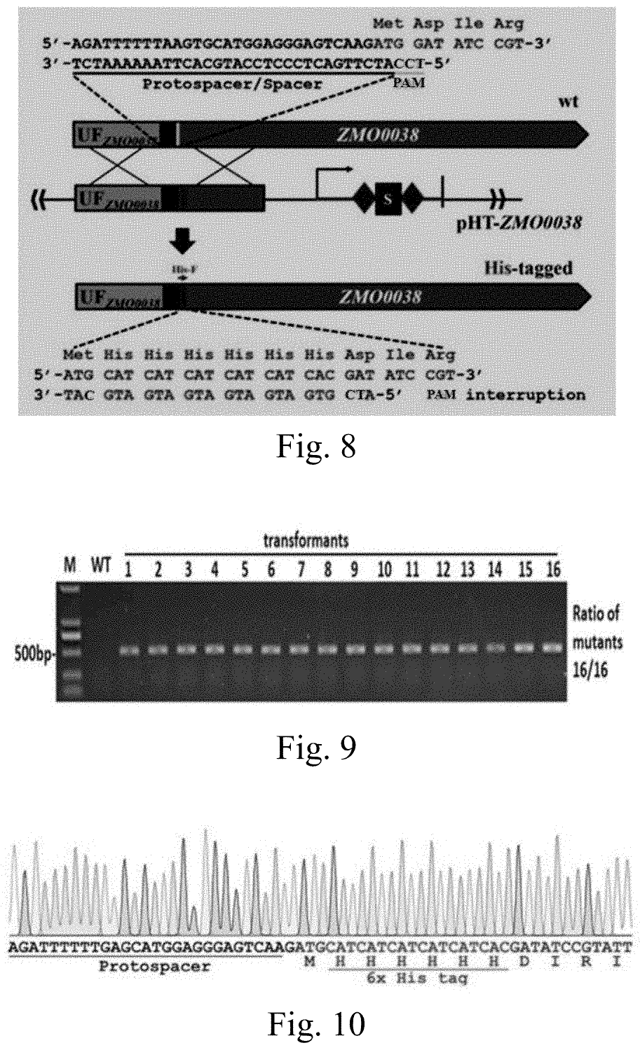

Similarly, for other types of gene insertion, the CRISPR-Cas system in Example 1 can also be selected. The following description takes the example of inserting a His-Tag tag after the ATG of the ZMO0038 gene start code as an example, so that the encoded protein can be purified through a nickel column. This method can be extended to any protein to be purified or investigated. The principle is shown in FIG. 8.

From the target gene sequence ZMO0038 near the start code ATG, the 32 bp sequence immediately downstream of 5′-TCC-3′ is intercepted as gRNA, and this sequence can be located on any strand of the genome. The sequences of ZMO0038 (His-Tag)-guide RNA primers are shown in SEQ ID NO: 18-SEQ ID NO: 19.

The gRNA primer was constructed on the vector: the plasmid containing the artificial CRISPR expression unit is linearized with Bsa I, annealed to the gRNA primer, and then ligated with T4 DNA ligase. The plasmid is electroporated into E. coli DH5α, and the transformants are then verified by colony PCR and Sanger sequencing. Experimental conditions are the same as in Example 3. The verification primers are: pEZ15A-F and 0038His-gRNA-R. The sequences are shown in SEQ ID NO: 11 and SEQ ID NO: 19, respectively. The constructed plasmids are all verified by sequencing.

The donor DNA sequence of the point mutation of the His-Tag tag is the DNA sequence with the His-Tag introduced by the primer design. The upstream and downstream arms of the donor DNA are selected to be point mutation of the His-Tag tag. The upstream and downstream positions are about 300 bp for each sequence, which is amplified and linked by fusion PCR technology. The vector constructed in the previous step is double digested with Xba I and EcoR I, and then transformed with donor DNA sequence by Gibson assembly method to E. coli DH5α, and then the transformants were verified by colony PCR. The editing plasmids constructed were all verified by sequencing. Experimental conditions are the same as in Example 3. His-Tag tag sequence: catcatcatcatcatcac, see SEQ ID NO:20. The sequences of donor DNA primers are shown in SEQ ID NO: 21-SEQ ID NO: 24.

The editing plasmid was electroporated into ZM4 competent cells, and positive clones were selected. The method is the same as in Example 3. The primer sequences are shown in SEQ ID NO: 25-SEQ ID NO: 26.

The results of colony PCR screening for positive clones are shown in FIG. 9. The results show that the efficiency of point mutation has reached 100%, and from another aspect, the invention is confirmed to be an efficient gene editing method. Using the above PCR products for sequencing analysis, the results are shown in FIG. 10. The sequencing results are compared with the wild-type strain genomic sequence, and it is found that the His-Tag tag insertion method was completely in accordance with the experimental design scheme, further illustrating the accuracy of the invention.

### Example 5 Application of Z. mobilis CRISPR Genome Editing System in Point Mutation

1. Select the genome editing system according to the target gene and its editing type. In this example, ZMO1237 is used as the target site, point mutation is performed on it, and the sequence of 23 bp downstream of the PAM site TTTN site is selected from the target gene as a guide in the construction of the target plasmid RNA targeting guide sequence. The forward primer is 5′-AGAT+(target sequence)-3′, and the reverse primer is 5′-TGAC+(target sequence complementary sequence)-3′. The CRISPR-Cas12a system described in Example 2 is selected. The sequence of the gRNA primer is shown in SEQ ID NO: 97-SEQ ID NO: 98.

2. Construction of Target Plasmid

The gRNA primer sequence was ligated to the editing plasmid containing the CRISPR expression unit prepared in Example 2: First, the vector was linearized with restriction enzyme Bsa I, and then the gRNA primer pair was annealed. The annealed product and the linearized vector were ligated using T4 DNA ligase and then transferred into E. coli DH5α. The recombinant is screened by colony PCR and finally verified by sequencing. The specific operation process was the same as in Example 3.

3. Designing of Donor DNA

Using single-stranded nucleotide (ssDNA) as a repair template to modify the broken part, and at the same time changing two bases near the target site to introduce Pst I digestion it. The length of ssDNA is 59-nt, which is complementary to the trailing strand encoding DNA. The sequence of ssDNA is shown in SEQ ID NO:99.

4. Transformation of the editing plasmid, about 200 ng of editing plasmid and 1 μg of ssDNA were transformed into competent cells containing the recombinant bacteria of Cas12a and cultured. Experimental methods and conditions are the same as in Example 3.

5. Screening of Recombinant Strains

The colony PCR detection conditions are the same as in Example 2, and the detection primer sequences are shown in SEQ ID NO: 100-SEQ ID NO: 101.

Recover the DNA fragment, and digest the DNA with the restriction enzyme Pst I, digestion system (DNA fragment, 200 ng; 10×buffer, 1 μL; Pst I, 0.2 μL; supplement H₂O to 10 μL. The conditions are the same as in Example 3). The fact that the DNA fragment can be cut correctly means that it is edited correctly, and the recombinant strain is further verified by sequencing.

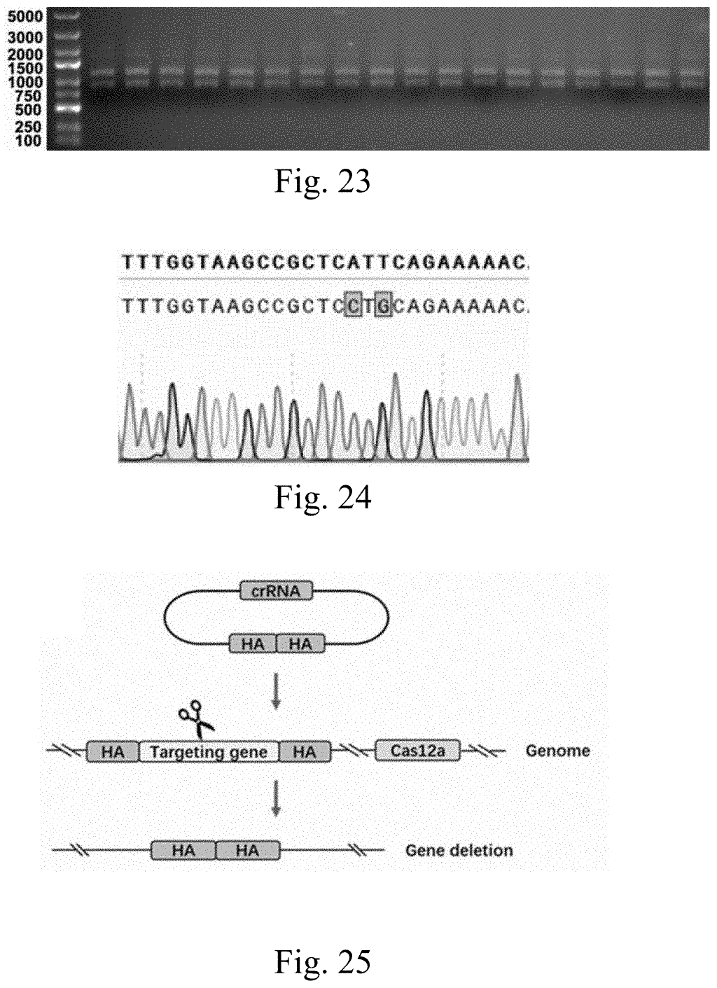

The results of electrophoresis and sequencing of digestion products are shown in FIG. 23 and FIG. 24. The results show that CRISPR-Cas12a can efficiently replace the bases in the genome with the help of ssDNA, and it is consistent with the design, and its efficiency can be as high as 100%.

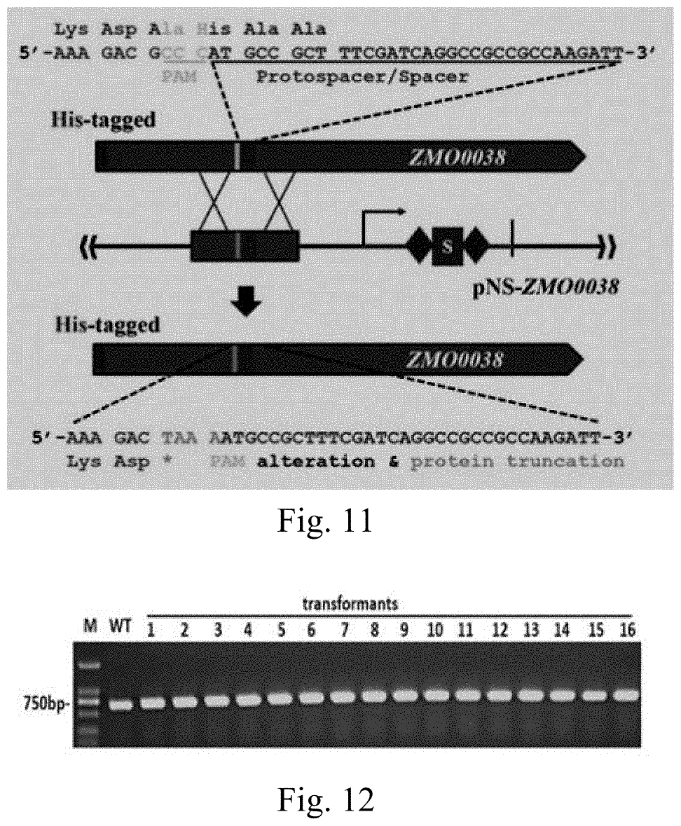

Similarly, for other types of point mutations, the CRISPR-Cas system in Example 1 can also be selected. The following takes the ZMO0038 gene inserted a His-Tag tag in Example 4 as a target, and mutates a few base sequences in the coding region of its gene, thereby introducing the stop code 5′-AAA-3′ to the protein it encodes. This method can also be extended to the study of protein active sites. The principle is shown in FIG. 11.

From the target gene sequence His-ZMO0038 coding region 5′-CCC-3′ immediately downstream of the 32 bp sequence as guide RNA, the sequence can only be located on the coding strand. The sequences of ZMO0038(PM)-guide RNA primers are shown in SEQ ID NO:27-SEQ ID NO:28.

The plasmid containing the artificial CRISPR expression unit was linearized with Bsa I, annealed with the gRNA primer, and then ligated with T4 DNA ligase. The plasmid was amplified by transformation into E. coli DH5α, and then the colonies were verified by colony PCR. Experimental methods are the same as in Example 3. The verification primers are: pEZ15A-F and 0038PM-gRNA-R. The plasmids constructed are all verified by sequencing.

The donor DNA sequence of site-directed mutagenesis is a base sequence with mutations introduced by primer design. The upstream and downstream arms of the donor DNA are selected to have a sequence of about 300 bp each upstream and downstream of the site-directed mutagenesis site, and expanded by fusion PCR technology. The vector constructed in the previous step was double digested with Xba I and EcoR I, and then assembled with donor DNA sequence by Gibson assembly method before transforming into E. coli DH5α. The transformants were verified by colony PCR. The constructed editing plasmids are all verified by sequencing. Experimental methods are the same as in Example 3. The sequences of donor DNA primers are shown in SEQ ID NO:29-SEQ ID NO:32.

The edited plasmid was electroporated into ZM4 competent cells, and colony PCR and sequencing were performed under the same conditions as in Example 3. See SEQ ID NO: 16-SEQ ID NO: 33 for primer sequences.

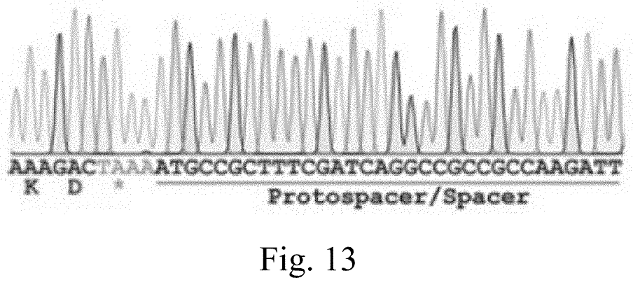

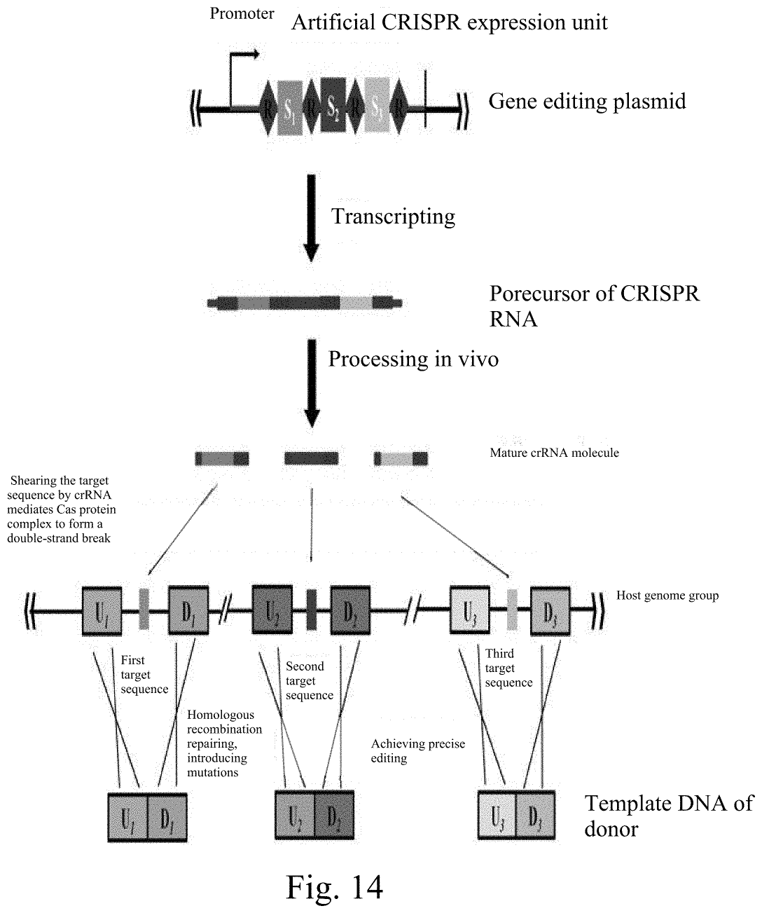

The electrophoresis results of PCR products are shown in FIG. 12, and the analysis of colony PCR product sequencing results is shown in FIG. 13. The results of the 16 samples submitted for sequencing are all mutant strains. The efficiency of site-directed mutation reached 100%, confirming that the invention is a highly efficient and accurate gene editing method.

### Example 6 Application of Z. mobilis CRISPR Genome Editing System in Simultaneous Editing of Multiple Gene Loci

1. The genome editing system is selected according to the target gene and its editing type, and the CRISPR-Cas system described in Example 1 is selected. The object of the multi-site gene editing in this embodiment is the CRISPR gene 1-4 (CRISPR1-4) encoded by the genome, where CRISPR3 and 4 are located close to each other on the genome, and they are used as an editing target for knockout.

From the 3′end of the target gene sequence CRISPR1-4, a 32-bp sequence immediately downstream of 5′-NCC-3′ is intercepted as a gRNA, which can be located on any strand of the genome.

The sequences of CRISPR1-gRNA, CRISPR2-gRNA and CRISPR3&4-gRNA are shown in SEQ ID NO:37-SEQ ID NO:39.

2. Cloning Artificial CRISPR Clusters into Editing Plasmids

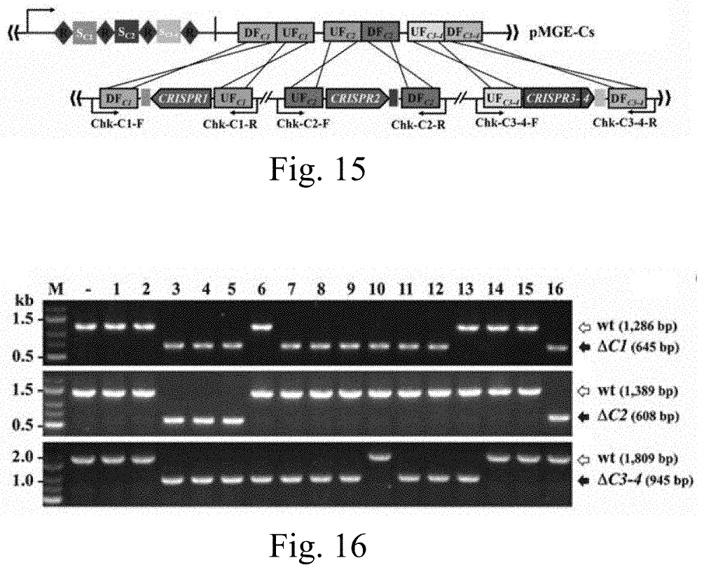

The cloning process is shown in FIG. 15. The gRNA of the above target gene was artificially ligated in series, and then assembled with the plasmid containing the artificial CRISPR expression unit prepared in Example 1 by Gibson assembly, and then the colonies of the transformants are verified by PCR. The constructed plasmids were verified by sequencing. Experimental methods are the same as in Example 1. The sequence of primers used for PCR verification of the colony is shown in SEQ ID NO: 40-SEQ ID NO: 41.

3. Cloning the Donor DNA Sequence into the Editing Plasmid

The sequences of about 300 bp on the upstream and downstream of the target gene were selected as the homologous recombination donor template DNA, which were amplified and ligated by fusion PCR technology, and ligated into the editing plasmid by Gibson assembly method. The products were then transformed into E. coli DH5a following by colony PCR verification and Sanger sequencing. Experimental conditions are the same as in Example 3. The sequences of donor DNA primers are shown in SEQ ID NO: 42-SEQ ID NO: 53.

4. Electroporate the editing plasmids into Z. mobilis ZM4 competent cells, then use primers CRISPR1-check-F and CRISPR1-check-R; CRISPR2-check-F and CRISPR2-check-R; CRISPR3&4-check-F and CRISP3&4-check-R to conduct colony PCR to screen positive colonies, respectively. The method is the same as in Example 3. The sequence of colony PCR screening primers is shown in SEQ ID NO: 54-SEQ ID NO: 59.

5. Perform colony PCR verification on the obtained transformants, screen positive clones, and further verify the PCR products by sequencing. The method is the same as in Example 3.

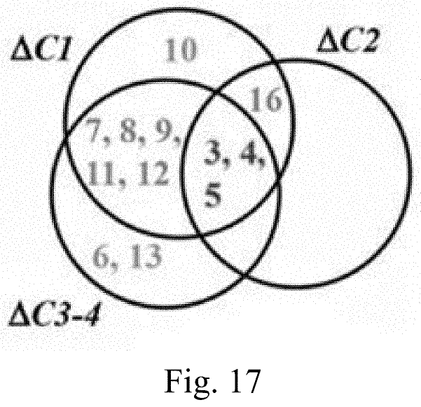

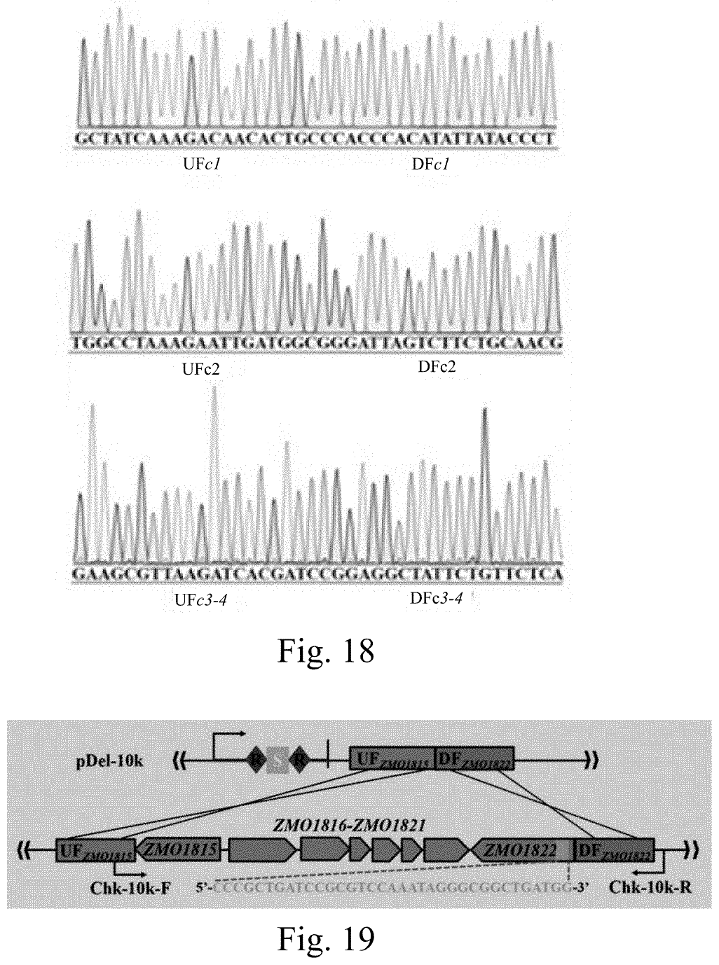

The results are shown in FIGS. 16-18. Among the 16 transformants tested, at least one target site editing accounted for 75%, and samples 3, 4 and 5 were successfully knocked out of 3 target genes at the same time. The results of sequencing analysis of the strains at the site indicate that all sites have been accurately edited according to the experimental design, which shows the high efficiency, accuracy and stability of the present invention in the application of gene editing.

### Example 7 Application of Z. mobilis CRISPR Genome Editing System in Efficient Deletion of Large Genome Fragments

In this embodiment, first, the bioinformatics method is used to determine the essential genes that need to be retained and the unnecessary genes that can be deleted, and a non-essential gene with a length of 10 kb is selected as the target knockout large segment. Then the gRNA and donor DNA sequences were designed. Finally, the artificial CRISPR cluster expression module and donor DNA sequence are constructed into the plasmid, and the plasmid is electroporated into Z. mobilis cells to complete the gene editing. The CRISPR-Cas system described in Example 1 was selected. The schematic diagram of the principle is shown in FIG. 19, and the specific experimental scheme is as follows:

(1) Selection of Target Knockout Large Segment

Through bioinformatics analysis, an unnecessary gene ZMO1815-ZMO1822 (10,021 bp) was found on the genome of the bacterium, which can be used as the target sequence for knocking out large fragments.

(2) Selection of gRNA of the Targeted Genes

From the target gene sequence ZMO1815-ZMO1822, any 32 bp sequence immediately downstream of 5′-CCC-3′ is intercepted as gRNA, which can be located on any strand of the genome. The sequences of gRNA primers for 10-kb genomic fragment deletion are shown in SEQ ID NO: 60-SEQ ID NO: 61.

(3) Construction of gRNA into Gene Editing Plasmid

The plasmid containing the artificial CRISPR expression unit prepared in Example 1 was linearized with Bsa I, annealed with gRNA primers, connected with T4 DNA ligase, and then transformed into E. coli DH5α. The transformants were then screened by colony PCR, and the candidate plasmids are further verified by Sanger sequencing. Experimental conditions are the same as in Example 3. Colony PCR verification primer sequence, see SEQ ID NO: 62-SEQ ID NO: 10.

(4) Acquisition of Donor DNA Sequence and its Construction on Editing Plasmid

The donor DNA sequence selects the sequence of about 1-Kb on the upstream and downstream of the target gene, which is amplified and connected by fusion PCR technology. The vector constructed in the previous step is digested with Xma I and Sac I (except for the type of enzymes used in the digestion system, other conditions and conditions are the same as in Example 1). The transformants were then screened by colony PCR, and the candidate plasmids are further verified by Sanger sequencing. Experimental conditions are the same as in Example 3. The sequences of donor DNA primers are shown in SEQ ID NO:63-SEQ ID NO:66.

(5) The editing plasmids were transformed into ZM4 competent cells, and primers 10K-check-F (SEQ ID NO: 67) and 10K-check-R (SEQ ID NO: 68) were used to screen positive transformants using colony PCR, which were further confirmed by Sanger sequencing. The method is the same as in Example 3.

(6) Results and Analysis

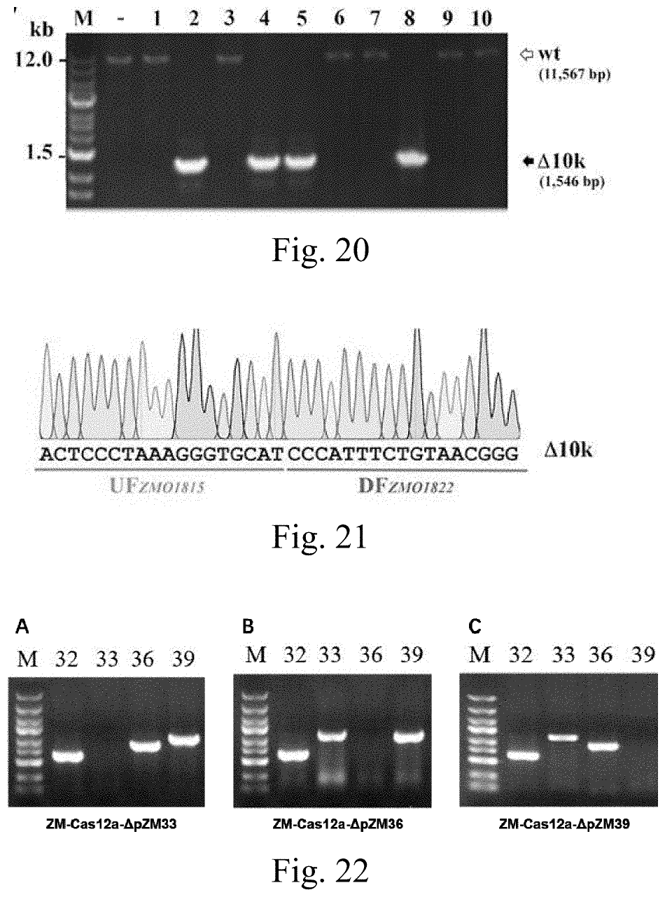

The results of colony PCR screening for positive clones are shown in FIG. 20. The results show that the 10-kb genome fragment was successfully knocked out, and the editing efficiency reached 40%, which shows that the invention can be used to efficiently delete large genome fragments. The above PCR products were sequenced and analyzed, and the results are shown in FIG. 21. In the obtained editing strain, the deletion of the gene cluster is completely consistent with the experimental design, which illustrates the accuracy of the invention in gene editing.

### Example 8 Application of Z. mobilis CRISPR Genome Editing System in the Elimination of Endogenous Plasmids 1. Selection of Target Sites

The genomic data of Z. mobilis ZM4 has been published, which contains 4 endogenous plasmids, and named pZM32 (32,791 bp), pZM33 (33,006 bp), pZM36 (36,494 bp) and pZM39 (39,266 bp) according to the sequence size. Sequence analysis showed that the four endogenous plasmids containing corresponding replication enzymes. If the replication enzyme is inactivated, the endogenous plasmid will lose the ability to replicate, and the endogenous plasmid will be eliminated from the strain.

In this embodiment, the sequence of 23 bp downstream of the TTTN of the PAM site is selected from the replicase gene of the endogenous plasmid as the target guide sequence for constructing the gRNA in the target plasmid to guide the cleavage of the target site by the nuclease. The forward primer is 5′-AGAT+(target sequence)-3′, and the reverse primer is 5′-TGAC+(target sequence complementary sequence)-3′. The CRISPR-Cas12a system described in Example 2 was selected.

The gRNA primer sequences of the four endogenous plasmids are shown in SEQ ID NO: 83-SEQ ID NO:90.

2. Construction of Target Plasmid

The gRNA primer sequences were used to construct the editing plasmid vector containing the CRISPR expression unit prepared in Example 2: First, the vector is linearized with the restriction enzyme Bsa I, and then the gRNA primer pair is annealed. The annealed product and the linearized vector are ligated with T4 DNA ligase, and then transformed into E. coli DH5a by a general chemical transformation method in the art for plasmid construction, wherein recombinants are screened by colony PCR and finally verified by sequencing. Experimental conditions are the same as in Example 3.

3. The target plasmid is then electro-porated into competent cells of the Z. mobilis recombinant strain containing the Cas12a, wherein the method is identical to Example 3.

4. Screening of Recombinant Strains

After the transformants grow out, colony PCR was performed to screen candidate recombinant strains using their specific primers, respectively. The PCR system and procedure are the same as in Example 2. The detection primers are shown in SEQ ID NO: 91-SEQ ID NO: 96.

The electrophoresis results of PCR products are shown in FIG. 22. The results show that three endogenous plasmids have been successfully eliminated, fully demonstrating that this method is an efficient method for eliminating endogenous plasmids, which provides a means for analyzing the function of plasmids and performing genome reduction.

The above are only the preferred embodiments of the present invention and are not intended to limit the present invention. Any modification, equivalent replacement and improvement made within the spirit and principle of the present invention should be included in the protection of the present invention within range.

1. A CRISPR-Cas system based on Zymomonas mobilis (Z. mobilis), comprising: four CRISPR structural sequences and one cas gene cluster, wherein the cas gene cluster comprises a cas1 gene, a cast-3 gene, a csy1 gene, a csy2 gene, a csy3 gene and a csy4 gene, wherein the cast-3 gene is in a fusion form fused by a cast gene and a cas3 gene.

2: A genome editing system for Z. mobilis using the exogenous CRISPR-Cas12a system comprising: an editing plasmid carrying guide RNA (gRNA) sequence, an artificial CRISPR expression unit, and donor DNA sequence, and a recombinant Z. mobilis strain containing an inducible nuclease Cas12a in its genome.

3. The genome editing system for exogenous CRISPR-Cas12a system based on the Z. mobilis, as recited in claim 2, wherein the recombinant Z. mobilis containing inducible nuclease Cas12a is obtained by integrating exogenous nuclease Cas12a into Z. mobilis containing the CRISPR-Cas system, and the expression of nuclease Cas12a is controlled by a tetracycline-inducible promoter.

4. The genome editing system for the exogenous CRISPR-Cas12a system based on the Z. mobilis, as recited in claim 2, wherein the artificial CRISPR expression unit comprises a constitutive promoter PJ23119, a repeat sequence and two restriction sites.

5. The genome editing system for exogenous CRISPR-Cas12a system based on the Z. mobilis, as recited in claim 2, wherein the PAM sequence of the editing system is TTTN.

6. A genome editing system for endogenous type I-F CRISPR-Cas system based on the Z. mobilis, comprising: an editing plasmid carrying a gRNA sequence, an artificial CRISPR expression unit and donor DNA sequence.

7. The genome editing system for endogenous type j-F CRISPR-Cas system based on the Z. mobilis, as recited in claim 6, wherein the artificial CRISPR expression unit comprises a leader sequence, a CRISPR cluster and a terminator; the CRISPR cluster comprises two enzyme cleavage sites inserted between two repeat sequences.

8. The genome editing system for endogenous type I-F CRISPR-Cas system based on the Z. mobilis, as recited in claim 7, wherein the leader sequence, CRISPR cluster and terminator sequence are shown in SEQ ID NO: 34-SEQ ID NO: 36 respectively

9. The genome editing system for endogenous type_I-F CRISPR-Cas system based on the Z. mobilis, as recited in claim 6, wherein the PAM sequence of the editing system is NCC.

10. (canceled)


SEQUENCE LISTING (123 sequences)

SEQ ID NO: 1 - DNA, 41 bp, Artificial sequence
  synthesized in laboratory
aattcccgatcgcgggcaacggtttattcagctatccgcgc

SEQ ID NO: 2 - DNA, 41 bp, Artificial sequence
  synthesized in laboratory
ctaggcgcggatagctgaataaaccgttgcccgcgatcggg

SEQ ID NO: 3 - DNA, 40 bp, Artificial sequence
  synthesized in laboratory
aattaaagatcgcgggcaacggtttattcagctatccgcg

SEQ ID NO: 4 - DNA, 40 bp, Artificial sequence
  synthesized in laboratory
ctaggcgcggatagctgaataaaccgttgcccgcgatctt

SEQ ID NO: 5 - DNA, 39 bp, Artificial sequence
  synthesized in laboratory
aattcccgtctggctgaaatgaggtccgacgatttgcat

SEQ ID NO: 6 - DNA, 39 bp, Artificial sequence
  synthesized in laboratory
gttaatgcaaatcgtcggacctcatttcagccagacggg

SEQ ID NO: 7 - DNA, 39 bp, Artificial sequence
  synthesized in laboratory
aattaaagtctggctgaaatgaggtccgacgatttgcat

SEQ ID NO: 8 - DNA, 39 bp, Artificial sequence
  synthesized in laboratory
gttaatgcaaatcgtcggacctcatttcagccagacttt

SEQ ID NO: 9 - DNA, 36 bp, Artificial sequence
  synthesized in laboratory
gaaaatgccgctttcgatcaggccgccgccaagatt

SEQ ID NO: 10 - DNA, 36 bp, Artificial sequence
  synthesized in laboratory
gaacaatcttggcggcggcctgatcgaaagcggcat

SEQ ID NO: 11 - DNA, 22 bp, Artificial sequence
  synthesized in laboratory
ggcaaagccaccctatttttag

SEQ ID NO: 12 - DNA, 46 bp, Artificial sequence
  synthesized in laboratory
aggtcaccagctcaccgtctgaattcccgaccatttgtatgacccg

SEQ ID NO: 13 - DNA, 40 bp, Artificial sequence
  synthesized in laboratory
tctatctgaatatttaacgacttgactccctccatgcact

SEQ ID NO: 14 - DNA, 40 bp, Artificial sequence
  synthesized in laboratory
agtgcatggagggagtcaagtcgttaaatattcagataga

SEQ ID NO: 15 - DNA, 46 bp, Artificial sequence
  synthesized in laboratory
ctcgagagatctgatatcactctagaatcggttctttcaggcggac

SEQ ID NO: 16 - DNA, 20 bp, Artificial sequence
  synthesized in laboratory
gaattattgcgtcacggcgc

SEQ ID NO: 17 - DNA, 20 bp, Artificial sequence
  synthesized in laboratory
gcgcatcacgagagccagcc

SEQ ID NO: 18 - DNA, 36 bp, Artificial sequence
  synthesized in laboratory
gaaaatcttgactccctccatgcacttaaaaaatct

SEQ ID NO: 19 - DNA, 36 bp, Artificial sequence
  synthesized in laboratory
gaacagattttttaagtgcatggagggagtcaagat

SEQ ID NO: 20 - DNA, 18 bp, Artificial sequence
  synthesized in laboratory
catcatcatcatcatcac

SEQ ID NO: 21 - DNA, 46 bp, Artificial sequence; identical to SEQ ID NO: 12
  synthesized in laboratory

SEQ ID NO: 22 - DNA, 41 bp, Artificial sequence
  synthesized in laboratory
gtgatgatgatgatgatgcatcttgactccctccatgcact

SEQ ID NO: 23 - DNA, 41 bp, Artificial sequence
  synthesized in laboratory
atgcatcatcatcatcatcacgatatccgtatttccggcca

SEQ ID NO: 24 - DNA, 46 bp, Artificial sequence
  synthesized in laboratory
ctcgagagatctgatatcactctagacggcaatgacgggaggatga

SEQ ID NO: 25 - DNA, 21 bp, Artificial sequence
  synthesized in laboratory
atgcatcatcatcatcatcac

SEQ ID NO: 26 - DNA, 20 bp, Artificial sequence
  synthesized in laboratory
aaacagcatcggaaacgctg

SEQ ID NO: 27 - DNA, 36 bp, Artificial sequence; identical to SEQ ID NO: 9
  synthesized in laboratory

SEQ ID NO: 28 - DNA, 36 bp, Artificial sequence; identical to SEQ ID NO: 10
  synthesized in laboratory

SEQ ID NO: 29 - DNA, 46 bp, Artificial sequence
  synthesized in laboratory
aggtcaccagctcaccgtctgaattcaacaaaatctaggtttctga

SEQ ID NO: 30 - DNA, 44 bp, Artificial sequence
  synthesized in laboratory
ggcctgatcgaaagcggcattttagtctttgtctatgccgcctg

SEQ ID NO: 31 - DNA, 44 bp, Artificial sequence
  synthesized in laboratory
caggcggcatagacaaagactaaaatgccgctttcgatcaggcc

SEQ ID NO: 32 - DNA, 46 bp, Artificial sequence
  synthesized in laboratory
ctcgagagatctgatatcactctagagcgacctctctcccgtttat

SEQ ID NO: 33 - DNA, 20 bp, Artificial sequence
  synthesized in laboratory
aaacagcatcggaaacgctg

SEQ ID NO: 34 - DNA, 148 bp, Artificial sequence
  synthesized in laboratory
tttgaccctttatttgaccctctttttttggcatgtaaaaaaatcctttaaaatcaataggttaaaaataggctctatttttagggttatttggctatttttgcccgatattcctttcatttagggggatttttaattatttactcta

SEQ ID NO: 35 - DNA, 72 bp, Artificial sequence
  synthesized in laboratory
gttcactgccgcacaggcagcttagaaaggagaccgaggtctcagttcactgccgcacaggcagcttagaaa

SEQ ID NO: 36 - DNA, 28 bp, Artificial sequence
  synthesized in laboratory
ggctcaccttcgggtgggcctttctgcg

SEQ ID NO: 37 - DNA, 32 bp, Artificial sequence
  synthesized in laboratory
tgtggggatatgatcggcatcggttttgataa

SEQ ID NO: 38 - DNA, 32 bp, Artificial sequence
  synthesized in laboratory
tctttaaaaccaccccaatgcaataaaatcta

SEQ ID NO: 39 - DNA, 32 bp, Artificial sequence
  synthesized in laboratory
aaccatgaaacagaacaacacttaaataagta

SEQ ID NO: 40 - DNA, 22 bp, Artificial sequence
  synthesized in laboratory
ggcaaagccaccctatttttag

SEQ ID NO: 41 - DNA, 32 bp, Artificial sequence
  synthesized in laboratory
tacttatttaagtgttgttctgtttcatggtt

SEQ ID NO: 42 - DNA, 46 bp, Artificial sequence
  synthesized in laboratory
aggtcaccagctcaccgtctgaattcatgccgccgccaataataac

SEQ ID NO: 43 - DNA, 40 bp, Artificial sequence
  synthesized in laboratory
agggtataatatgtgggtgggcagtgttgtctttgatagc

SEQ ID NO: 44 - DNA, 40 bp, Artificial sequence
  synthesized in laboratory
gctatcaaagacaacactgcccacccacatattataccct

SEQ ID NO: 45 - DNA, 40 bp, Artificial sequence
  synthesized in laboratory
cagatatgccgcaacaggccggcaggatgtatctattagg

SEQ ID NO: 46 - DNA, 40 bp, Artificial sequence
  synthesized in laboratory
cctaatagatacatcctgccggcctgttgcggcatatctg

SEQ ID NO: 47 - DNA, 40 bp, Artificial sequence
  synthesized in laboratory
cgttgcagaagactaatcccgccatcaattctttaggcca

SEQ ID NO: 48 - DNA, 40 bp, Artificial sequence
  synthesized in laboratory
tggcctaaagaattgatggcgggattagtcttctgcaacg

SEQ ID NO: 49 - DNA, 40 bp, Artificial sequence
  synthesized in laboratory
aatctcaatatatttgagcgggctttggtgtcgcttttct

SEQ ID NO: 50 - DNA, 40 bp, Artificial sequence
  synthesized in laboratory
agaaaagcgacaccaaagcccgctcaaatatattgagatt

SEQ ID NO: 51 - DNA, 40 bp, Artificial sequence
  synthesized in laboratory
tgagaacagaatagcctccggatcgtgatcttaacgcttc

SEQ ID NO: 52 - DNA, 40 bp, Artificial sequence
  synthesized in laboratory
gaagcgttaagatcacgatccggaggctattctgttctca

SEQ ID NO: 53 - DNA, 46 bp, Artificial sequence
  synthesized in laboratory
ctcgagagatctgatatcactctagaggcttcttatggttaaaata

SEQ ID NO: 54 - DNA, 20 bp, Artificial sequence
  synthesized in laboratory
caggatgccgccgccaataa

SEQ ID NO: 55 - DNA, 20 bp, Artificial sequence
  synthesized in laboratory
gggcgatcgcagcgatgaaa

SEQ ID NO: 56 - DNA, 20 bp, Artificial sequence
  synthesized in laboratory
ggcctgttgcggcatatctg

SEQ ID NO: 57 - DNA, 20 bp, Artificial sequence
  synthesized in laboratory
tattggcggcgtgcagcaaa

SEQ ID NO: 58 - DNA, 20 bp, Artificial sequence
  synthesized in laboratory
ggcaaaattgttggcggcta

SEQ ID NO: 59 - DNA, 21 bp, Artificial sequence
  synthesized in laboratory
tcgcaaccagaatgacatgcg

SEQ ID NO: 60 - DNA, 36 bp, Artificial sequence
  synthesized in laboratory
gaaagaccttatgcctatgtcgatacaaccacgaat

SEQ ID NO: 61 - DNA, 36 bp, Artificial sequence
  synthesized in laboratory
gaacattcgtggttgtatcgacataggcataaggtc

SEQ ID NO: 62 - DNA, 22 bp, Artificial sequence
  synthesized in laboratory
ggcaaagccaccctatttttag

SEQ ID NO: 63 - DNA, 46 bp, Artificial sequence
  synthesized in laboratory
tcgagcgtcccatagatctcgagctcggttgtgataagcggcagat

SEQ ID NO: 64 - DNA, 40 bp, Artificial sequence
  synthesized in laboratory
aaacccgttacagaaatgggatgcaccctttagggagtcg

SEQ ID NO: 65 - DNA, 40 bp, Artificial sequence
  synthesized in laboratory
cgactccctaaagggtgcatcccatttctgtaacgggttt

SEQ ID NO: 66 - DNA, 46 bp, Artificial sequence
  synthesized in laboratory
gggtcaaataaagggtcaaacccgggcgagttcatattcacggtcg

SEQ ID NO: 67 - DNA, 20 bp, Artificial sequence
  synthesized in laboratory
gacaagagcggaatccgcgt

SEQ ID NO: 68 - DNA, 20 bp, Artificial sequence
  synthesized in laboratory
ggatggtgcctttccctgaa

SEQ ID NO: 69 - DNA, 3903 bp, Artificial sequence
  synthesized in laboratory
atgtcaatttatcaagaatttgtgaacaaatatagcctgagcaaaaccctgcgttttgaactgattccgcagggtaaaaccctggaaaacattaaagcacgtggtctgattctggatgatgaaaaacgtgccaaagactacaaaaaagccaaacaaatcatcgataaataccaccagttcttcatcgaagaaattctgagcagcgtttgcattagcgaagatctgctgcagaattattccgacgtttatttcaaactgaaaaaaagcgacgatgataacctgcagaaagatttcaaaagcgccaaagataccatcaaaaaacaaattagcgagtatatcaaagacagcgagaaattcaaaaacctgttcaaccagaatctgatcgatgccaaaaaaggtcaagaaagcgatctgatcctgtggctgaaacagagcaaagataatggcatcgaactgtttaaagccaacagcgatattaccgatattgatgaagcactggaaatcatcaaaagctttaaaggttggaccacctactttaaaggctttcacgaaaatcgcaaaaacgtgtatagcagcaatgatattccgaccagcattatctatcgcatcgttgatgataatctgcctaaatttctggaaaataaagccaaatatgaaagcctgaaagacaaagcaccggaagcaattaactatgagcagatcaaaaaagatctggccgaagaactgacctttgacattgattacaaaaccagcgaagttaaccagcgtgtttttagcctggatgaagtttttgaaattgccaacttcaacaactacctgaatcagagcggtatcaccaaattcaataccattatcggtggcaaattcgtgaatggcgaaaataccaaacgcaaaggcatcaacgaatacattaatctgtatagccagcagattaacgataaaacgctgaaaaaatacaaaatgagcgtgctgttcaaacaaattctgtcagataccgaaagcaaaagcttcgtgattgacaaactggaagatgatagtgatgttgttaccaccatgcagagcttttatgaacaaatcgcagcgtttaaaaccgtggaagagaaatccattaaagaaaccctgagcctgctgtttgatgatctgaaagcacagaaactggacctgtccaaaatctacttcaaaaacgataaatccctgaccgatctgagccagcaggttttcgatgattatagcgttattggcaccgcagttctggaatatatcacacagcagattgcaccgaaaaatctggataatccgagcaaaaaagaacaagagctgatcgccaaaaaaaccgagaaagcgaaatatctgagcctggaaacaattaaactggccctggaagaatttaacaaacaccgcgacattgataaacagtgccgctttgaagaaatcctggcaaattttgcagcaatcccgatgatctttgatgaaattgcgcagaataaagataacctggcacagatcagcatcaaatatcagaatcagggaaaaaaagacctgctgcaagcaagtgccgaagatgatgttaaagcgattaaagatctgctggatcagaccaataacctgctgcataaactgaaaatctttcacattagccagagcgaggataaagcgaacattctggataaagatgagcacttctatctggtgtttgaagagtgttattttgagctggcaaatattgtgccgctgtataacaaaatccgcaactatattacccagaaaccgtatagcgacgaaaaattcaaactgaactttgagaatagcaccctggccaatggttgggataaaaacaaagaaccggataataccgccatcctgttcattaaagatgataaatactatctgggcgtgatgaacaaaaaaaacaacaaaatcttcgacgataaagccatcaaagagaataaaggcgaaggttacaaaaaaatcgtgtacaaactgctgcctggtgcgaataaaatgctgccgaaagtgttttttagcgccaaatccatcaaattctataacccgagcgaagatattctgcgtattcgtaatcatagcacccataccaaaaatggtagtccgcagaaaggctatgaaaaattcgagttcaacattgaggattgccgcaaattcatcgacttctacaaacagtccattagcaaacatccggaatggaaagactttggttttcgttttagcgatacccagcgctataacagcattgatgaattttatcgcgaagtggaaaaccagggctataaactgacatttgaaaacatcagcgagagctatattgatagcgttgtgaatcagggtaaactgtacctgtttcagatctataacaaagactttagcgcctatagcaaaggtcgtccgaatctgcataccctgtattggaaagcactgttcgatgaacgtaatctgcaggatgttgtctacaaactgaatggtgaagcagaactgttttatcgcaaacagagtatcccgaaaaaaatcacccatccggcaaaagaagcaatcgcgaacaaaaacaaagataacccgaaaaaagaaagcgtgttcgagtatgatctgatcaaagataaacgcttcaccgaagataaattctttttccattgcccgatcaccatcaactttaaaagcagcggtgcgaacaaattcaacgatgaaatcaatctgctgctgaaagaaaaagccaacgatgttcatattctgagcattgatcgtggtgaacgtcatctggcctattacaccctggttgatggtaaaggcaatattatcaaacaggacaccttcaacattatcggcaatgatcgtatgaaaaccaactaccatgataaactggcagccattgaaaaagatcgtgatagcgcacgtaaagattggaaaaaaatcaacaacattaaagaaatgaaagaaggctacctgagccaggttgttcatgaaatcgccaaactggtgattgaatataatgccattgtggtgttcgaggatctgaacttcggtttcaaacgtggtcgtttcaaagttgagaaacaggtgtatcaaaaactggaaaaaatgctgatcgaaaaactgaattacctggtgttcaaagacaacgaattcgataaaaccggtggtgttctgcgtgcatatcagctgaccgcaccttttgaaaccttcaaaaaaatgggtaaacagaccggcatcatctattatgttccggcaggttttacctccaaaatttgtccggttaccggctttgttaatcagctgtatccgaaatatgagagcgttagcaaaagccaagagtttttcagcaaatttgataaaatctgctataacctggacaaaggctactttgaattcagctttgactataaaaactttggcgataaagcagccaaaggcaaatggaccattgcaagctttggtagccgtctgattaactttcgtaacagcgacaaaaaccataactgggatacccgtgaagtttatccgaccaaagagctggaaaaactgctgaaagattacagcattgaatatggtcatggcgaatgtattaaagccgcaatttgtggtgagtccgacaaaaaattctttgcaaaactgaccagcgtgctgaataccattctgcagatgcgtaatagcaaaaccggcaccgaactggattatctgattagtccggttgcagatgtgaacggcaattttttcgatagccgtcaggctccgaaaaatatgccgcaggatgcagatgcaaatggtgcctatcatattggcctgaaaggtctgatgctgctgggtcgcattaaaaacaatcaagaaggcaaaaaactgaacctggtgatcaaaaacgaagagtattttgagttcgtgcagaataggaataactaa

SEQ ID NO: 70 - DNA, 736 bp, Artificial sequence
  synthesized in laboratory
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaagtattcaaatgatctaaagaggagaaaggatctccc

SEQ ID NO: 71 - DNA, 51 bp, Artificial sequence
  synthesized in laboratory
gaggagaaaggatctcccatgtcaatttatcaagaatttgtgaacaaatat

SEQ ID NO: 72 - DNA, 39 bp, Artificial sequence
  synthesized in laboratory
ctggcgtcgggcgtgataaaacgaaaggcccagtctttc

SEQ ID NO: 73 - DNA, 24 bp, Artificial sequence
  synthesized in laboratory
ttaagacccactttcacatttaag

SEQ ID NO: 74 - DNA, 23 bp, Artificial sequence
  synthesized in laboratory
gggagatcctttctcctctttag

SEQ ID NO: 75 - DNA, 39 bp, Artificial sequence
  synthesized in laboratory
ctgaatatttaacgaaattctcatgtttgacagcttatc

SEQ ID NO: 76 - DNA, 38 bp, Artificial sequence
  synthesized in laboratory
gaaagtgggtcttaaattcagtactcactacggaattg

SEQ ID NO: 77 - DNA, 21 bp, Artificial sequence
  synthesized in laboratory
ttaggcgagaagggaaagggc

SEQ ID NO: 78 - DNA, 27 bp, Artificial sequence
  synthesized in laboratory
tcgttaaatattcagatagacggagat

SEQ ID NO: 79 - DNA, 16 bp, Artificial sequence
  synthesized in laboratory
tcacgcccgacgccag

SEQ ID NO: 80 - DNA, 35 bp, Artificial sequence
  synthesized in laboratory
ctcgagtttggatcccaccctctggtgattgtcga

SEQ ID NO: 81 - DNA, 28 bp, Artificial sequence
  synthesized in laboratory
ggatccaaactcgagtaaggatctccag

SEQ ID NO: 82 - DNA, 34 bp, Artificial sequence
  synthesized in laboratory
atgtatatctccttcttaaaagatcttttgaatt

SEQ ID NO: 83 - DNA, 27 bp, Artificial sequence
  synthesized in laboratory
agatgtcaaagcgatatcggacaataa

SEQ ID NO: 84 - DNA, 27 bp, Artificial sequence
  synthesized in laboratory
tgacttattgtccgatatcgctttgac

SEQ ID NO: 85 - DNA, 27 bp, Artificial sequence
  synthesized in laboratory
agatcccccgatatttctttcatgaat

SEQ ID NO: 86 - DNA, 27 bp, Artificial sequence
  synthesized in laboratory
tgacattcatgaaagaaatatcggggg

SEQ ID NO: 87 - DNA, 27 bp, Artificial sequence
  synthesized in laboratory
agatgagcgtgtgatgagccggaggaa

SEQ ID NO: 88 - DNA, 27 bp, Artificial sequence
  synthesized in laboratory
tgacttcctccggctcatcacacgctc

SEQ ID NO: 89 - DNA, 27 bp, Artificial sequence
  synthesized in laboratory
agatggccgaacaaaatgaggttaaaa

SEQ ID NO: 90 - DNA, 27 bp, Artificial sequence
  synthesized in laboratory
tgacttttaacctcattttgttcggcc

SEQ ID NO: 91 - DNA, 24 bp, Artificial sequence
  synthesized in laboratory
agcttgatccatcacccatattac

SEQ ID NO: 92 - DNA, 17 bp, Artificial sequence
  synthesized in laboratory
tctaccattgcggcgtc

SEQ ID NO: 93 - DNA, 24 bp, Artificial sequence
  synthesized in laboratory
tcgcatctgccttgtttgtataag

SEQ ID NO: 94 - DNA, 25 bp, Artificial sequence
  synthesized in laboratory
atgaatactgcgcagaatcagactc

SEQ ID NO: 95 - DNA, 20 bp, Artificial sequence
  synthesized in laboratory
attactgaggaaccgcctcg

SEQ ID NO: 96 - DNA, 20 bp, Artificial sequence
  synthesized in laboratory
tgcgattccaggatatacgg

SEQ ID NO: 97 - DNA, 27 bp, Artificial sequence
  synthesized in laboratory
agatgtaagccgctcattcagaaaaac

SEQ ID NO: 98 - DNA, 27 bp, Artificial sequence
  synthesized in laboratory
tgacgtttttctgaatgagcggcttac

SEQ ID NO: 99 - DNA, 59 bp, Artificial sequence
  synthesized in laboratory
gaacattatggccatgaccttgtttttctgcaggagcggcttaccaaagagacagcaga

SEQ ID NO: 100 - DNA, 45 bp, Artificial sequence
  synthesized in laboratory
tagggtgaggttatagctatgaaaaaagtcaatcgtattgcagtg

SEQ ID NO: 101 - DNA, 42 bp, Artificial sequence
  synthesized in laboratory
gctttttcaaaaagcttattacaatatcggtgccattgtttc

SEQ ID NO: 102 - DNA, 27 bp, Artificial sequence
  synthesized in laboratory
agatccgcagcgggttattctgatcaa

SEQ ID NO: 103 - DNA, 27 bp, Artificial sequence
  synthesized in laboratory
tgacttgatcagaataacccgctgcgg

SEQ ID NO: 104 - DNA, 34 bp, Artificial sequence; identical to SEQ ID NO: 82
  synthesized in laboratory

SEQ ID NO: 105 - DNA, 28 bp, Artificial sequence
  synthesized in laboratory
ggatccaaactcgagtaaggatctccag

SEQ ID NO: 106 - DNA, 38 bp, Artificial sequence
  synthesized in laboratory
ttactcgagtttggatcctctgcgcctattgattacgg

SEQ ID NO: 107 - DNA, 31 bp, Artificial sequence
  synthesized in laboratory
tatcgagccttttacttaaaataatctatcg

SEQ ID NO: 108 - DNA, 22 bp, Artificial sequence
  synthesized in laboratory
gctttttgaaaaagccgtttcc

SEQ ID NO: 109 - DNA, 34 bp, Artificial sequence
  synthesized in laboratory
taagaaggagatatacatgcagcggatcaaggcg

SEQ ID NO: 110 - DNA, 20 bp, Artificial sequence
  synthesized in laboratory
atctgcgcctattgattacg

SEQ ID NO: 111 - DNA, 22 bp, Artificial sequence
  synthesized in laboratory
tcaagatttcaaacaagcggtc

SEQ ID NO: 112 - DNA, 27 bp, Artificial sequence
  synthesized in laboratory
agatccgcagcgggttattctgatcaa

SEQ ID NO: 113 - DNA, 27 bp, Artificial sequence
  synthesized in laboratory
tgacttgatcagaataacccgctgcgg

SEQ ID NO: 114 - DNA, 34 bp, Artificial sequence; identical to SEQ ID NO: 82
  synthesized in laboratory

SEQ ID NO: 115 - DNA, 28 bp, Artificial sequence
  synthesized in laboratory
ggatccaaactcgagtaaggatctccag

SEQ ID NO: 116 - DNA, 38 bp, Artificial sequence; identical to SEQ ID NO: 106
  synthesized in laboratory

SEQ ID NO: 117 - DNA, 31 bp, Artificial sequence; identical to SEQ ID NO: 107
  synthesized in laboratory

SEQ ID NO: 118 - DNA, 22 bp, Artificial sequence
  synthesized in laboratory
gctttttgaaaaagccgtttcc

SEQ ID NO: 119 - DNA, 34 bp, Artificial sequence; identical to SEQ ID NO: 109
  synthesized in laboratory

SEQ ID NO: 120 - DNA, 42 bp, Artificial sequence
  synthesized in laboratory
taagtaaaaggctcgatatcgatcaacaacccgaatcctatc

SEQ ID NO: 121 - DNA, 46 bp, Artificial sequence
  synthesized in laboratory
acggctttttcaaaaagcttatttatacaattcatccataccgccg

SEQ ID NO: 122 - DNA, 20 bp, Artificial sequence
  synthesized in laboratory
atctgcgcctattgattacg

SEQ ID NO: 123 - DNA, 22 bp, Artificial sequence
  synthesized in laboratory
tcaagatttcaaacaagcggtc